(19) United States
(12) Patent Application Publication
(10) Pub. No.: US 2008/0124801 A1
(43) Pub. Date: May 29, 2008

(54) Pluripotent cell growth media

(75) Inventors: Joe Mee, Edinburgh (GB); Helen Bradburn, Edinburgh (GB)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Stem Cell Sciences (UK) Ltd., Edinburgh (GB)

(21) Appl. No.: 11/825,068

(22) Filed: Jul. 3, 2007

(30) Foreign Application Priority Data
Nov. 27, 2006 (GB) GB 0623635.0

(51) Int. Cl. C12N 15/87; C12N 5/06

(52) U.S. Cl. 435/455; 435/389; 435/404

(57) ABSTRACT

Self renewal of pluripotent cells in culture is promoted using a serum-free medium that comprises, inter alia, insulin and progesterone and has an osmolarity of 260-270 Osm/kg.

This application claims the benefit under 35 U.S.C. § 119 of Great Britain Application No. 0623635.0, filed on Nov. 27, 2006.

The present invention relates to culture conditions and methods of culturing pluripotent stem cells in order to promote stem cell self renewal and to prevent or control differentiation of the stem cells. The invention further provides methods for isolating and maintaining homogeneous preparations of pluripotent stem cells. The methods and compositions provided are suitable for culturing and isolating pluripotent stem cells such as embryonic stem (ES) cells.

The establishment and maintenance of in vitro pluripotent stem cell cultures in the presence of medium containing serum and Leukemia Inhibitory Factor (LIF) is well known (Smith et al. (1988) Nature 336: 688-90). Such methods have been used to maintain pluripotent embryonic stem (ES) cells from permissive strains of mice over many passages. Maintenance and self renewal of pluripotent stem cell cultures is further supported where the stem cells are cultured in the presence of feeder cells or extracts thereof, usually mouse fibroblast cells. Under such conditions it is possible to maintain human ES cells in a pluripotent state over many passages in culture.

However, a continuing problem in this field is that, despite intense efforts, it remains the case that pluripotent cultures of ES cells can be derived and maintained for extended periods from only a few species and, even in those species, not from all embryos. In some cases, pluripotent cells can be identified but they cannot then be maintained in culture for sufficient time to enable study or genetic manipulation of the cells. This is particularly the case for human and rodent (other than some strains of mouse) cells.

A further problem is that ES cells that can indeed be maintained in a pluripotent state in culture over many passages can only be so maintained using medium that contains serum or serum extract, and hence is undefined, or alternatively, using cell culture conditions that require the presence of other cells, such as the fibroblast feeder cells used to maintain human ES cells. However, where ES cells are intended to be subjected to subsequent controlled differentiation into desired cell types, it is undesirable to utilize an undefined culture medium or to have heterologous cells present.

The serum typically used in culturing pluripotent stem cells is fetal calf (bovine) serum, which is known to contain a complex mixture of cytokines and other signaling molecules. In order to control differentiation pathways it is undesirable to introduce unknown cytokines to the culture medium whose influence on the eventual outcome of differentiation is unquantifiable, and could be potentially deleterious or otherwise undesirable. Further, each serum batch is unique and introduces variation into culture protocols.

As a result, the ES cells obtained by culture in such complex media, and any differentiated progeny thereof, risk being contaminated by components of the media and/or by cells such as feeder cells that are required to maintain the ES cells. These factors militate against development of good manufacturing practices for therapeutic and other applications of ES cells and their progeny.

While a number of groups have reported use of serum-free media, growth of pluripotent cells has been disappointing or not consistent over repeated passages. Better media giving improved growth is needed.

When deriving a differentiated cell population from an ES cell culture, it is desirable to be able to convert a high proportion of the ES cells into progeny of the same type—i.e., to maintain as homogeneous a population of cells as possible. However, in practice, it is observed that, following differentiation, a cell population is obtained that contains a heterogenous mixture of cells. Hence, it is desirable to be able to carry out differentiation of an ES cell population and obtain a purer population of progeny.

EP 1077254 describes methods and compositions for the differentiation of stromal cells from adipose tissue, which may include interleukins, FGF and serum, and amounts of TGF-β sufficient to induce differentiation into smooth muscle.

EP 0753574 describes methods and compositions for ex vivo human progenitor cell expansion. The culture medium contains stromal cells, typically transformed fibroblast cells.

WO 00/05344 describes maintenance of Drosophila germline stem cells and propagation of somatic stem cells of other species when co-cultured with genetically engineered Drosophila cells.

WO 96/40866 describes serum-free culture of human haematopoietic progenitor and stem cells in a culture medium containing at least one of a peptone, a protease inhibitor and a pituitary extract.

US 2002/0028510 describes methods and compositions for the differentiation of pluripotent cells from umbilical cord blood into neuronal cell types.

U.S. Pat. No. 5,750,376 describes methods and compositions for differentiation of multipotent neural stem cells in culture medium supplemented with at least one growth factor.

Wiles and Johansson, Exp. Cell Research, 1999 (247) pgs 241-248 describe maintenance of undifferentiated cell lines in the presence of LIF and Fetal Bovine Serum. When ES cells were grown in a serum free medium they rapidly lost their ES cell phenotype and developed into a range of cell types, including neuroectoderm.

Hence, one embodiment of the invention provides methods of culturing and culture media suitable for pluripotent stem cells that are capable of supporting self-renewal of said stem cells in an undifferentiated state for many passages. An other embodiment provides a culturing system that permits maintenance of a pluripotent stem cell culture in vitro until differentiation of the cells is induced in a controlled manner. A further embodiment provides methods and compositions that enhance the isolation of pluripotent stem cells and facilitate their isolation from organisms refractory to ES cell isolation or from which pluripotent stem cells have not yet been isolated.

The present invention is based, in part, on the observation that culturing pluripotent stem cells, such as ES cells, in a serum-free medium comprising a mixture of insulin and progesterone promotes self renewal of the stem cells for multiple passages. The invention is further based on the observation that a medium comprising a combination of insulin, progesterone, sodium putrescine, sodium selenite and apotransferrin is capable of promoting self renewal of stem cells in culture. It has further been established that the osmolarity of the medium can significantly influence whether the medium serves to either promote self renewal of the stem cells or differentiation.

An advantage of the present culture system is that differentiation of ES cells is reduced compared to culture in the presence of serum. This is significant because often the most pluripotent ES cells tend to differentiate considerably in serum, making their manipulation and expansion problematic.

The invention provides both media that is optimized for the growth of human pluripotent cells and media that is optimized for the growth of mouse (and other non-human, mammalian) pluripotent cells.

Accordingly, a first aspect of the invention provides a pluripotent cell culture medium comprising:
(a) insulin; and
(b) progesterone,

wherein the medium is free of serum.

In a second aspect, the invention provides a pluripotent cell culture medium comprising:
(a) insulin;
(b) progesterone; and
(c) an iron transporter, such as, e.g., transferrin and/or apotransferrin,

wherein the medium is free of serum.

A third aspect of the invention provides a serum-free medium for self-renewal of pluripotent cells, preferably human pluripotent cells, comprising:
(a) a basal medium;
(b) insulin; and
(c) progesterone.

In one embodiment, the various media of the invention comprise insulin at a concentration of 5 to 30 mg/L, a concentration of 10 to 20 mg/L, a concentration of 11 to 14 mg/L, or a concentration of about 12.9 or 12.5 mg/L. Progesterone may be present in the culture media at a concentration of 0.005 to 0.05 mg/L, a concentration of 0.01 to 0.03 mg/L, or a concentration of about 0.011 or 0.0099 mg/L. When present, the concentration of transferrin and/or apotransferrin in the culture media may be from 25 to 75 mg/L, or from 40 to 60 mg/L, or about 50 mg/L.

In certain embodiments, the insulin and/or progesterone and/or transferrin/apotransferrin are obtained from a recombinant source. In some embodiments, when culturing human pluripotent cells, the media incorporates the human forms of these proteins. Insulin and progesterone represent factors which, inter alia, promote cell survival and/or metabolism of the cells.

The culture media defined above may additionally comprise putrescine and/or sodium putrescine.2HCl. In one embodiment, putrescine is present at a concentration of from 3 to 20 mg/L, a concentration of 5 to 15 mg/L, a concentration of 7 to 12 mg/L, or a concentration of about 8 or about 9.6 mg/L.

In yet a further embodiment, sodium putrescine is present at a concentration of from 0.0005 to 0.1 mg/L. In some embodiments, when culturing human pluripotent cells, sodium putrescine is present in the medium at a concentration of 0.03 to 0.05 mg/L or at a concentration of about 0.04 mg/L. In some embodiments, when culturing mouse or other non-human mammalian pluripotent cells, sodium putrescine is present in the medium at a concentration of from 0.0005 to 0.0051 mg/L, or a concentration of about 0.001 mg/L.

The culture media of the invention may also comprise sodium selenite at, e.g., a concentration of from 1×10-6 to 0.01 mg/L. In some embodiments, when the medium is optimized for culture of human pluripotent cells, the sodium selenite is present in the medium at a concentration of from 2×10-6 to 3×10-6 mg/L, such as, e.g. a concentration of 2.5×10-6 mg/L. In some embodiments, when the medium is optimized for culture of mouse (and other non-human mammalian) pluripotent cells, the sodium selenite is present at a concentration of from 0.001 to 0.01 mg/L or at a concentration of about 0.002 mg/L.

Preferably, the culture media of the invention has an osmolarity of from 260 to 270 Osm/kg, 263 to 266 Osm/kg, or about 265 Osm/kg.

When used to culture human pluripotent cells, in certain embodiments the culture medium additionally comprises basic Fibroblast Growth Factor (bFGF) at, e.g., a concentration of from 0.005 to 0.1 mg/L, a concentration of from 0.008 to 0.05 mg/L, or a concentration of about 0.01 mg/L. In some embodiments the bFGF is the human form of the protein (e.g., obtained by recombinant means). When used to culture mouse cells, in some embodiments, the media is additionally supplemented with B27. For mouse cells the culture medium may also contain (i) an activator of the signaling pathway downstream from a receptor of the TGF-β superfamily (e.g. a bone morphogenic protein, e.g. BMP-4) and (ii) an activator of a gp130 signaling pathway (e.g. LIF or IL-6 and sIL-6R).

Media used to culture human cells do not require supplementation either with B27 or with any of components (i) and (ii) as described above.

In one embodiment the culture media defined above also comprise serum albumin (e.g., at a concentration of from 10 to 100 mg/L, a concentration of 20 to 60 mg/L, or a concentration of about 37.5 mg/L). Serum albumin can be used in purified or recombinant form, and if recombinant this has the advantage of absence of potential contaminating factors, e.g. cytokines, etc. When using the medium to culture human pluripotent cells, in some embodiments the serum albumin is human serum albumin (HSA).

The culture media of the invention are free of serum. In addition, these media may also be prepared such that they are free of serum extract, free of feeder cells and free of feeder cell extract.

The media of the invention may incorporate a basal medium. Basal medium is medium that supplies essential sources of carbon and/or vitamins and/or minerals for the ES cells. The basal medium is generally free of protein and incapable on its own of supporting self-renewal of ES cells. Examples of basal media suitable for use in the present invention include DMEM F12, neurobasal medium, and combinations thereof.

In some embodiments, the media of the invention incorporate an iron transporter. The iron transporter provides a source of iron or provides the ability to take up iron from the culture medium. Suitable iron transporters include transferrin and apotransferrin.

In a specific embodiment, the medium of the invention is one that is fully defined. Such a medium does not contain any components which are undefined, that is to say components whose content is unknown or which may contain undefined or varying factors that are unspecified. An advantage of using a fully defined medium is that efficient and consistent protocols for culture and subsequent manipulation of pluripotent cells can be derived. Further, it is found that maintenance of cells in a pluripotent state is achievable with higher efficiency and greater predictability and that when differentiation is induced in cells cultured using a defined medium the response to the differentiation signal is more homogenous then when undefined medium is used.

A medium according to the present invention may be used for culture of pluripotent stem cells from any adult tissue.

The media of the invention are preferably free of animal components. By that it is meant that the medium does not contain any components which have been purified from animals, particularly from animal serum and the like. Instead, such media use components which are not directly obtained from animals. This can be achieved for example, by using recombinant means to generate protein components of the medium. The absence of the animal components has the particular advantage of avoiding contamination of the medium with undefined animal derived components, or with potential infectious agents associated with animal products.

In preferred embodiments, the media of the invention as defined above are used to culture human pluripotent stem cells, and may additionally comprise in any combination, one or more, two or more, three or more, four or more, five or more, six or more, seven or more, eight or more, nine or more, ten or more etc, or all of the following components:

100 to 200 mg/L or about 159 mg/L of calcium chloride anhydrous;

0.0005 to 0.0008 mg/L or about 0.00065 mg/L of cupric sulfate;

0.05 to 1.00 mg/L or about 0.075 mg/L of ferric nitrate;

0.01 to 0.03 mg/L or about 0.021 mg/L of ferric sulfate;

200 to 400 mg/L or about 307 mg/L of potassium chloride;

10 to 20 mg/L or about 14.4 mg/L of magnesium chloride;

40 to 100 mg/L or about 63.2 mg/L of magnesium sulfate;

3000 to 7000 mg/L or about 5021 mg/L sodium chloride;

500 to 1500 mg/L or about 1100 mg/L sodium bicarbonate;

50 to 150 mg/L or about 94 mg/L sodium phosphate monobasic;

20 to 50 mg/L or about 36 mg/L sodium phosphate dibasic;

0.10 to 0.30 mg/L or about 0.22 mg/L zinc sulphate;

3000 to 5000 mg/L or about 3836 mg/L D-glucose;

5 to 12 mg/L or about 8 mg/L phenol red;

2000 to 4000 mg/L or about 3099 mg/L HEPES;

0.7 to 2.0 mg/L or about 1.2 mg/L sodium hypoxanthine;

0.01 to 0.30 mg/L or about 0.021 mg/L linoleic acid;

0.025 to 0.075 mg/L or about 0.05 mg/L DL-68-thiotic acid;

20 to 70 mg/L or about 40 mg/L sodium pyruvate;

2 to 5 mg/L or about 3.2 mg/L alanine;

50 to 200 mg/L or about 116 mg/L arginine;

2 to 7 mg/L or about 4.2 mg/L asparagine;

2 to 7 mg/L or about 3.3 mg/L aspartic acid;

5 to 15 mg/L or about 9.4 mg/L cysteine.H₂O;

10 to 20 mg/L or about 15.8 mg/L cystine.2HCl;

200 to 400 mg/L or about 293 mg/L glutamine;

2 to 7 mg/L or about 3.7 mg/L glutamic acid;

20 to 30 mg/L or about 24 mg/L glycine;

20 to 60 mg/L or about 36 mg/L histidine;

50 to 100 mg/L or about 80 mg/L isoleucine;

50 to 100 mg/L or about 82 mg/L leucine;

100 to 150 mg/L or about 119 mg/L lysine;

10 to 40 mg/L or about 23 mg/L methionine;

25 to 75 mg/L or about 50 mg/L phenylalanine;

5 to 20 mg/L or about 12 mg/L proline;

15 to 50 mg/L or about 34 mg/L serine;

50 to 100 mg/L or about 74 mg/L threonine;

5 to 20 mg/L or about 12 mg/L tryptophan;

40 to 90 mg/L or about 64 mg/L tyrosine.2Na.2H₂O;

50 to 100 mg/L or about 73 mg/L valine;

0.0010 to 0.0030 mg/L or about 0.0018 mg/L biotin;

1 to 4 mg/L or about 3.1 mg/L D-calcium panthenate;

4 to 8 mg/L or about 6.5 mg/L choline chloride;

1 to 5 mg/L or about 3.3 mg/L folic acid;

5 to 15 mg/L or about 9.9 mg/L i-inositol;

1 to 5 mg/L or about 3.08 mg/L niacinamide;

1 to 5 mg/L or about 3.02 mg/L pyridoxine HCl;

0.1 to 0.5 mg/L or about 0.31 mg/L riboflavine;

2 to 5 mg/L or about 3.1 mg/L thiamine HCI;

0.1 to 0.4 mg/L or about 0.18 mg/L thymidine; and/or

0.20 to 0.75 mg/L or about 0.51 mg/L vitamin B12.

In some embodiments, the media of the invention as defined herein are used to culture mouse and other non-human pluripotent stem cells, and may additionally comprise in any combination, one or more, two or more, three or more, four or more, five or more, six or more, seven or more, eight or more, nine or more, ten or more etc, or all of the following components:

100 to 200 mg/L or about 154 mg/L of calcium chloride;

1 to 2 mg/L or about 1.4 mg/L of calcium chloride anhydrous;

0.0005 to 0.0020 mg/L or about 0.0010 mg/L of cupric sulfate;

0.04 to 0.08 mg/L or about 0.051 mg/L of ferric nitrate;

0.3 to 0.5 mg/L or about 0.42 mg/L of ferric sulfate;

200 to 400 mg/L or about 316 mg/L of potassium chloride;

0.2 to 0.4 mg/L or about 0.34 mg/L of magnesium chloride;

40 to 80 mg/L or about 61 mg/L of magnesium chloride anhydrous;

0.4 to 0.8 mg/L or about 0.58 mg/L of magnesium sulfate;

50 to 150 mg/L or about 100 mg/L of magnesium sulfate anhydrous;

3000 to 7000 mg/L or about 5633 mg/L sodium chloride;

1000 to 4000 mg/L or about 2438 mg/L sodium bicarbonate;

0.5 to 0.9 mg/L or about 0.74 mg/L sodium phosphate monobasic (Na₂HPO₄.H₂O);

50 to 100 mg/L or about 71 mg/L sodium phosphate monobasic (Na₂HPO₄.2H₂O);

50 to 100 mg/L or about 72 mg/L sodium phosphate dibasic;

0.8 to 1.5 mg/L or about 1.2 mg/L sodium hydroxide;

0.2 to 0.6 mg/L or about 0.46 mg/L zinc sulphate;

2000 to 5000 mg/L or about 3188 mg/L D-glucose;

5 to 10 mg/L or about 8 mg/L phenol red;

2000 to 4000 mg/L or about 3021 mg/L HEPES;

0.01 to 0.04 mg/L or about 0.028 mg/L sodium hypoxanthine;

0.01 to 0.3 mg/L or about 0.142 mg/L linoleic acid;

0.05 to 0.15 mg/L or about 0.101 mg/L DL-68-thiotic acid;

20 to 70 mg/L or about 58 mg/L sodium pyruvate;

10 to 16 mg/L or about 13.6 mg/L alanine;

50 to 200 mg/L or about 149 mg/L arginine;

15 to 25 mg/L or about 20 mg/L asparagine;

0.05 to 0.10 mg/L or about 0.09 mg/L asparagine.H₂O;

15 to 25 mg/L or about 20 mg/L aspartic acid;

10 to 25 mg/L or about 18 mg/L cysteine.HCl;

0.1 to 0.3 mg/L or about 0.2 mg/L cysteine.HCl.H₂O;

20 to 30 mg/L or about 24 mg/L cystine;

0.1 to 0.5 mg/L or about 0.37 mg/L cystine.2HCl;

250 to 450 mg/L or about 367 mg/L glutamine;

15 to 30 mg/L or about 22 mg/L glutamic acid;

15 to 25 mg/L or about 19.7 mg/L glycine;

20 to 60 mg/L or about 32 mg/L histidine;

40 to 90 mg/L or about 55 mg/L isoleucine;

40 to 90 mg/L or about 59.7 mg/L leucine;

80 to 120 mg/L or about 92 mg/L lysine;

10 to 40 mg/L or about 17.5 mg/L methionine;

20 to 60 mg/L or about 36 mg/L phenylalanine;

20 to 40 mg/L or about 30 mg/L proline;

15 to 50 mg/L or about 37 mg/L serine;

30 to 80 mg/L or about 54 mg/L threonine;

5 to 20 mg/L or about 9 mg/L tryptophan;

20 to 50 mg/L or about 39 mg/L tyrosine;

0.5 to 0.8 mg/L or about 0.66 mg/L tyrosine.2Na.2H₂O;

30 to 70 mg/L or about 53 mg/L valine;

0.010 to 0.020 mg/L or about 0.014 mg/L biotin;

2 to 4 mg/L or about 2.277 mg/L D-calcium panthenate;

7 to 12 mg/L or about 9.1 mg/L choline chloride;

1 to 4 mg/L or about 2.7 mg/L folic acid;

10 to 15 mg/L or about 12.8 mg/L i-inositol;

1 to 3 mg/L or about 2.04 mg/L niacinamide;

1 to 3 mg/L or about 2.05 mg/L pyridoxine HCl;

0.1 to 0.5 mg/L or about 0.22 mg/L riboflavine;

1 to 3 mg/L or about 2.18 thiamine HCl;

0.2 to 0.5 mg/L or about 0.354 mg/L thymidine;

0.50 to 1.00 mg/L or about 0.74 mg/L vitamin B12; and/or

0.05 to 0.2 mg/L or about 0.10 mg/L all-trans retinol.

In one embodiment of the invention the various media defined above may additionally comprise an agent that suppresses differentiation of pluripotent stem cells.

The various media of the invention may additionally comprise other defined factors which aid in maintaining and optimizing viability and self renewal of the cells in culture. For example, the media may comprise an agonist of a receptor of the TGF-β superfamily (such as BMP) and/or an activator of gp130 downstream signaling pathway (such as LIF). These components provide self renewal stimuli rather than pro-differentiation signals, and therefore help maintain the stem cells in a pluripotent state. ES cells can be maintained using the methods and culture media of the invention for a range of time, from several days (such as, e.g., 6 days) up to or longer than 1, 3, 6, 9 or 12 months, or longer. In some embodiments, the ES cells are maintained for 2, 3, 4, 5 or 6 weeks. ES cells can be maintained using the methods and culture media of the invention for a range of passage number, including from about 20 passages up to 40, 60, 80 or more passages in a culture. In some embodiments, the ES cells are maintained for 25, 30, or 35 passages. In other embodiments, the ES cells are maintained for 45 or 50 passages.

Other additional components which may be incorporated into the media of the invention include one or more of the following:
hormones such as corticosterone and T3;
retinyl acetate at, e.g., a concentration of about 0.01 mg/L;
trace elements such as ammonium metavanadate, cupric sulphate and manganous chloride;
β-mercaptoethanol at, e.g., a concentration of from 5 to 10 mg/L, or a concentration of about 7.8 mg/L;
L-carnitine at, e.g., a concentration of about 0.2 mg/L;
ethanolamine at, e.g., a concentration of about 0.1 mg/L;
D(+)-galactose at, e.g., a concentration of about 1.5 mg/L;
albumin;
albu-MAXall;
bovine serum albumin; and/or
antioxidants, including:
α-tocopherol at, e.g., a concentration of about 0.1 mg/L;
α-tocopherol acetate at, e.g., a concentration of about 0.1 mg/L;
catalase at, e.g., a concentration of about 1.6 mg/L;
glutathione at, e.g., a concentration of about 0.1 mg/L; and/or
superoxide dismutase at, e.g., a concentration of about 0.25 mg/L.

The media of the invention are useful in promoting self renewal of pluripotent cells. Accordingly, in a further aspect, the invention provides a use of a medium as defined above for promoting self renewal of pluripotent cells in culture.

As used herein, self renewal of pluripotent cells refers to their ability to be grown in culture and retain their pluripotency. A wide range of assays can be used to confirm the characteristics of the ES cells, including cell morphology, differentiation potential, and gene expression. For example, the pluripotency of ES cells can be confirmed by assaying the ability of the ES cells to differentiate into cell types representative of each of the three primary germ layers: ectoderm, endoderm, and mesoderm. In addition, the continued expression of one or more ES cell-specific markers such as Oct4 or Sox2 can be readily assayed. For example, as in Example 4, below, the continued expression of GFP by the Oct4-GFP ES cells indicates that the cells are still ES cells and therefore that the culture is undergoing self-renewal.

In one embodiment, the media of the invention includes an agent that suppresses differentiation, such as an inhibitor of the FGF receptor, for at least part of the culturing period to suppress the tendency of ES cells to differentiate. For example, ES cells can be cultured in a medium as defined above for a specified period before the FGF receptor inhibitor is removed. Suitable FGF receptor inhibitors include the compounds SU5402 and PD173074. Alternatively, a competitive inhibitor of the FGF receptor can be used, suitably a soluble form of the receptor.

In an alternative embodiment, the FGF receptor inhibitor is not removed from the medium. Hence, the FGF receptor inhibitor is present in the culture medium for an extended period. ES cells can be grown in culture for at least 20 passages in N2B27 medium in the presence of an FGF inhibitor. In some embodiments where the FGF receptor inhibitor is not removed from the medium, the FGF inhibitor is a specific inhibitor, i.e., it has little or no activity on other receptors.

In yet a further aspect, the invention provides a method of culturing pluripotent cells so as to promote cell self renewal, comprising maintaining the cells in a medium as defined above.

Methods of the invention can be used for stimulating self-renewal of ES cells in medium which is free of serum and free of serum extract, which cells have previously been passaged in the presence of serum or serum extract. In certain embodiments, such methods are also carried out in the absence of feeder cells and/or feeder cell extracts.

Accordingly, in another aspect, the invention provides a method of culturing pluripotent cells, comprising:
(a) maintaining the cells in a pluripotent state in culture, optionally on feeders, in the presence of serum or an extract of serum;
(b) passaging the pluripotent cells at least once;
(c) withdrawing the serum or the serum extract from the medium and withdrawing the feeders if present, so that the medium is free of feeders, serum and serum extract; and
(d) subsequently maintaining the cells in a pluripotent state in the presence of a medium as defined herein.

At around the time that the serum or extract of serum is withdrawn from the medium, it is an option to add to the medium an agent that suppresses differentiation, for example, an FGF-receptor inhibitor.

In a specific embodiment of this method, the pluripotent cells are cultured in the presence of an agent that suppresses differentiation, preferably wherein the agent that suppresses differentiation is added to culture medium at around the time that serum or serum extract is withdrawn.

In yet a further aspect, the invention provides a method of obtaining a transfected population of pluripotent cells, comprising:
(a) transfecting pluripotent cells with a construct encoding a selectable marker;
(b) plating the pluripotent cells;
(c) culturing the pluripotent cells in the presence of a medium as defined herein; and
(d) selecting for the pluripotent cells that express the selectable marker.

In one embodiment the selectable marker encodes an antibiotic resistance or cell surface marker.

Another aspect of the invention provides a method of culturing pluripotent cells, comprising:
(a) transferring an individual pluripotent cell to a culture vessel; and
(b) culturing the pluripotent cell in the presence of a medium as defined herein, so as to obtain a clonal population of pluripotent cells, all of which are the progeny of a single pluripotent cell.

Embryonic stem cells have been reported from a number of mammalian sources including mouse (Bradley et al (1984) Nature 309: 255-56), American mink (Mol Reprod Dev (1992) December; 33(4):418-31), pig and sheep (J Reprod Fertil Suppl (1991); 43:255-60), hamster (Dev Biol (1988) May; 127(1):224-7) and cow (Roux Arch Dev Biol (1992); 201: 134-141). It will be appreciated that the methods, uses and compositions of the present invention are suitable for adaptation to culturing of other mammalian pluripotent cell cultures, including human, primate and rodent (e.g. mouse), and avian ES cells.

Suitable cell densities for the methods and uses of the invention will vary according to the pluripotent stem cells being used and the nature of any desired progeny. Good results have been obtained by culturing embryonic stem cells in monolayer culture, dissociating the embryonic stem cells and subsequently culturing the embryonic stem cells in monolayer culture on a culture surface at a density of from 0.2-2.5×10⁴ cells per cm², more particularly at a density of from 0.5-1.5×10⁴ per cm². The cells proliferate as adherent monolayers.

Typical surfaces for culture of ES cells and their progeny according to the invention are culture surfaces recognized in this field as useful for cell culture, and these include surfaces of plastics, metal, composites, though commonly a surface such as a plastic tissue culture plate, widely commercially available, is used. Such plates are often a few centimeters in diameter. For scale up, this type of plate can be used at much larger diameters and many repeats plate units used.

It will be readily appreciated that the cells can be cultured in a variety of different culture vessels. For example, in one embodiment the culture vessel is an individual well on a plate.

It is further common for the culture surface to comprise a cell adhesion protein, usually coated onto the surface. Receptors or other molecules on the cells bind to the protein or other cell culture substrate and this promotes adhesion to the surface and it is suggested promotes growth. Gelatin coated plates are commonly available and are suitable for the invention, and other proteins may also be used.

Once a stable, homogenous culture of ES cells is obtained, the culture conditions can be altered to direct differentiation of the cells into one or more cell types selected from ectodermal, mesodermal or endodermal cell fates. Addition of, or withdrawal of cytokines and signalling factors, can enable the derivation of specific differentiated cell populations at high efficiency.

As discussed above, the culture medium of the invention is optionally supplemented with an inhibitor of differentiation of ES cells. Alternatively, when differentiation is desired, signalling factors that direct differentiation of ES cells towards a specific phenotype may be added.

Culture of cells is preferably carried out in an adherent culture, and in examples of the invention it has been found that following maintenance of cells in a pluripotent state, differentiation can be induced with a high degree of uniformity and with high cell viability. Adherent cultures may be promoted by the inclusion of a cell adhesion protein, and in specific examples of the invention gelatin has been used as a coating for the culture substrate.

In certain embodiments, pluripotent cells are cultured according to the invention in monolayer culture. In alternative embodiments cells may be grown in suspension culture or as pre-cell aggregates; cells can also be grown on beads or on other suitable scaffolds such as membranes or other 3-dimensional structures.

Yet another aspect of the invention provides a method of obtaining a differentiated cell comprising culturing a pluripotent cell as described and allowing or causing the cell to differentiate, wherein the cell contains a selectable marker which is capable of differential expression in the desired differentiated cell compared with other cell-types, including pluripotent stem cells, whereby differential expression of the selectable marker enables or results in preferential isolation and/or survival and/or division of the desired differentiated cells.

The differentiated cell can be a tissue stem or progenitor cell, and may be a terminally differentiated cell.

The media and methods of the invention are useful for promoting self renewal of pluripotent cells from multiple species. In particular, the media and methods of the invention can be used to culture human and mouse pluripotent cells.

Particular embodiments of the present invention are described below by way of the following examples. The examples are provided to illustrate embodiments of the invention but are not considered as limiting in any way.

### EXAMPLES

### Example 1

### A Serum-Free Medium for Culturing Human Pluripotent Cells

A medium for culturing human pluripotent cells in vitro was prepared. The composition of this medium, termed HEScGRO, is detailed in Table 1.

TABLE 1
Human Pluripotent Cell Animal-Component-Free Media

| | |
|---|---|
| Component | mg/L |

INORGANIC SALTS

| | | |
|---|---|---|
| Calcium Chloride Anhydrous | CaCl₃ | 158.695 |
| Cupric Sulfate | CuSO₄•5H₂O | 0.000654 |
| Ferric Nitrate | Fe(NO₃)•9H₂O | 0.0751 |
| Ferric Sulfate | FeSO₄•7H₂O | 0.0209 |
| Potassium Chloride | KCl | 306.969 |
| Magnesium Chloride | MgCl2 | 14.418 |
| Magnesium Sulfate | MgSO₄ | 63.237 |
| Sodium Chloride | NaCl | 5021.73 |
| Sodium Bicarbonate | NaHCO₄ | 1100 |
| Sodium Phosphate Monobasic | NaH₂PO₄•H₂O | 93.964 |
| Sodium Phosphate dibasic | Na₂HPO4 | 35.753 |
| | Na2HPO4•7H2O | |
| Zinc Sulfate | ZnSO₄•7H₂O | 0.217 |

OTHER COMPONENTS

| | |
|---|---|
| D-Glucose (Dexrose) | 3836.3 |
| Phenol Red | 8.127 |
| HEPES | 3099.505 |
| Na Hypoxanthine | 1.203 |
| Linoleic acid | 0.0211 |
| DL-68-Thioctic Acid | 0.0528 |
| Sodium Putrescine.2HCl | 0.0407 |
| Putrescine | 8 |
| Sodium Selenite | 2.5 × 10⁻⁶ |
| Sodium Pyruvate | 40.1885 |

AMINO ACIDS

| | |
|---|---|
| Alanine | 3.24 |
| Arginine.HCl | 116.255 |
| Asparagine | 4.19 |
| Aspartic acid | 3.347 |
| Cysteine.H₂O | 9.445 |
| Cystine.2HCl | 15.752 |
| Glutamic acid | 3.7 |
| Glutamine | 293.55 |
| Glycine | 24.439 |
| Histidine HCl•H₂O | 36.847 |
| Isoleucine | 79.921 |
| Leucine | 82.227 |
| Lysine HCl | 118.937 |
| Methionine | 23.679 |
| Phenylalanine | 50.861 |
| Proline | 12.564 |
| Serine | 34.214 |
| Threonine | 74.408 |
| Tryptophan | 12.54 |
| Tyrosine.2Na•2H₂O | 64.086 |
| Valine | 73.606 |

VITAMINS

| | |
|---|---|
| Biotin | 0.00176 |
| D-Calcium panthenate | 3.127 |
| Choline chloride | 6.52 |
| Folic acid | 3.334 |
| i-Inositol | 9.904 |
| Niacinamide | 3.079 |
| Pyridoxine HCl | 3.022 |
| Riboflavine | 0.31 |
| Thiamine HCl | 3.092 |
| Thymidine | 0.183 |
| Vitamin B12 | 0.512 |

PROTEINS

| | |
|---|---|
| Human recombinant Insulin | 12.5 |
| Human ApoTransferrin | 50 |
| Progesterone | 0.0099 |

OTHER

| | |
|---|---|
| Recombinant Human Serum Albumin | 0.18 |
| β-mercaptoethanol | 7.868 |
| Human recombinant bFGF | 0.04 |

The above medium was prepared in two batches, having respective osmolarities of 263 and 266 Osm/kg.

### Example 2

### A Serum-Free Medium for Culturing Mouse Pluripotent Cells

A medium suitable for the culture of mouse pluripotent cells was prepared. The composition of the medium, known specifically as EScGRO, is set out in Table 2.

TABLE 2
Mouse Pluripotent Cell Animal-Component-Free Media

| | |
|---|---|
| Component | mg/L |

INORGANIC SALTS

| | | |
|---|---|---|
| Calcium Chloride | CaCl2•2H2O | 154.000 |
| Calcium Chloride Anhydrous | | 1.385 |
| Cupric Sulfate | CuSO₄•5H₂O | 0.001 |
| Ferric Nitrate | Fe(NO₃)•9H₂O | 0.051 |
| Ferric Sulfate | FeSO₄•7H₂O | 0.420 |
| Potassium Chloride | KCl | 315.503 |
| Magnesium Chloride | MgCl2 | 0.340 |
| Magnesium chloride Andydrous | MgCl2•6H2O | 61.000 |
| Magnesium Sulfate | MgSO₄ | 0.580 |
| Magnesium Sulphate Anhydrous | MgSO4•7H2O | 100.000 |
| Sodium Chloride | NaCl | 5632.590 |
| Sodium Bicarbonate | NaHCO₃ | 2438.000 |
| Sodium Phosphate Monobasic | NaH₂PO₄•H₂O | 0.742 |
| Sodium Phosphate Monobasic | NaH₂PO₄•2H₂O | 70.500 |
| Sodium Phosphate dibasic | Na₂HPO4 | 71.844 |
| Sodium Hydroxyde | NaOH | 1.200 |
| Zinc Sulfate | ZnSO₄•7H₂O | 0.455 |

OTHER COMPONENTS

| | |
|---|---|
| D-Glucose | 3188.426 |
| Phenol Red | 8.196 |
| HEPES | 3021.457 |
| Na Hypoxanthine | 0.028 |
| Hypoxanthine | 2.000 |
| Linoleic acid | 0.142 |
| DL-68-Thioctic Acid | 0.101 |
| Lipoic acid | 0.000 |
| Sodium Putrescine.2HCl | 0.001 |
| Putrescine.2HCl | 0.081 |
| Putrescine | 9.610 |
| Sodium Selenite | 0.002 |
| Sodium Pyruvate | 57.500 |
| Thymidine | 0.354 |
| L-Carnitine | 0.200 |
| Ethanolamine | 0.100 |
| D(+)-Galactose | 1.500 |
| Linolenic acid | 0.100 |
| Bmercaptoethanol | 7.813 |
| BMP4* | 0.01 |
| LIF* | 0.01 |
| human recombinant bEGF** | 0.01 |

AMINO ACIDS

| | |
|---|---|
| Alanine | 13.603 |
| Arginine.HCl | 149.252 |
| Asparagine | 19.700 |
| Asparagine.H2O | 0.089 |
| Aspartic acid | 20.029 |
| Cysteine.HCl | 18.000 |
| Cysteine HCl•H₂O | 0.209 |
| Cystine | 24.000 |
| Cystine.2HCl | 0.372 |
| Glutamic acid | 22.137 |
| Glutamine | 367.335 |
| Glycine | 19.723 |
| Histidine HCl•H₂O | 31.874 |
| Isoleucine | 55.147 |
| Leucine | 59.701 |
| Lysine HCl | 92.334 |
| Methionine | 17.455 |
| Phenylalanine | 35.921 |
| Proline | 29.731 |
| Serine | 37.062 |
| Threonine | 54.135 |
| Tryptophan | 9.107 |
| Tyrosine | 38.700 |
| Tyrosine.2Na•2H₂O | 0.663 |
| Valine | 53.478 |

VITAMINS

| | |
|---|---|
| Biotin | 0.014 |
| D-Calcium panthenate | 2.277 |
| Choline chloride | 9.107 |
| Folic acid | 2.681 |
| i-Inositol | 12.750 |
| Niacinamide | 2.042 |
| Pyridoxine HCl | 2.054 |
| Riboflavin | 0.221 |
| Thiamine HCl | 2.176 |
| Vitamin B12 | 0.742 |
| Retinyl acetate | 0.010 |
| All Trans Retinol | 0.010 |

HORMONES

| | |
|---|---|
| bovine Insulin | 12.900 |
| Progesterone | 0.011 |

PROTEINS

| | |
|---|---|
| Hmn apo Transferrin | 50.500 |
| BovineSA | 287.500 |

ANTIOXIDANTS

| | |
|---|---|
| a-tocopherol (Vitamin E) | 0.100 |
| a-tocopherol, acetate | 0.100 |
| Catalase | 1.600 |
| Glutathione | 0.100 |
| Superoxide dismutase | 0.250 |

### Example 3

### Method for Growing ES Cells in Serum Free, Feeder Cell Free Defined Culture Media

ES cells are cultured in 0.1% gelatin coated dishes in HEScGRO or EScGRO medium as defined above. For passaging, a standard protein-free cell dissociation buffer is used to dissociate cells.

The plating density of the cells is approximately 1-5×10⁴/cm².

At the start of culture, the medium is further supplemented with SU5402 (5 μM) to suppress differentiation. Cells are transferred to media free of SU5402 after two passages.

ES cells are maintained in these serum free conditions for 20 passages over a three month period. Cells are normally passaged every 2-4 days depending on plating density. Occasionally, cell are passaged 7-10 days after plating at low clonal density.

The ES cells maintain pluripotency after multiple passages.

### Example 4

### Maintenance of Oct4-GFP Expression in ES Cells Cultured Under Serum Free Conditions

Oct4-GFP ES cells are cultured in HEScGRO or EScGRO medium as defined above in 0.1% gelatine coated plates. After four passages light microscope images are taken of the cells under phase contrast to show morphology and UV fluorescence to show expression of GFP.

It is envisaged that the cultured cells will maintain their pluripotent phenotype as indicated by both cells morphology and expression of GFP.

### Example 5

### Stable Transfection of ES Cells

E14 TG2A ES cells are cultured in a serum-free culture medium according to the invention. The cells are propagated on 0.1% gelatin coated plates, harvested and electroporated with pPCAG-tauGFP-IP. Transfected cells are replated on a 10 cm diameter dish at a density of 10⁵-10⁶ per dish. After 24 hours, 0.5 g/ml puromycin is added to select for positive colonies.

Between 8 and 10 days later, single GFP positive colonies are picked into each single well of a 96 well plate and the cells cultured in the same medium as described herein.

Stable transfection of the ES cells, determined by GFP fluorescence, and expansion of morphologically undifferentiated ES cells is envisaged.

### Example 6

### Clonal Self-Renewal of ES Cells

Individual ES cells are picked and transferred into wells of a 96 well plate, and cultured in HEScGRO or EScGRO medium as described for Examples 1 and 2 above.

The efficiency of cloning of these ES cells, previously grown in serum-free media for at least one passage, is expected to be similar to that obtainable using serum-containing medium. The clones are expected to grow and be passaged and grown further as undifferentiated ES cells.

In previous experiments (data not published) we have discovered that ES cells grown in serum-containing medium when transferred directly to a serum-free medium demonstrate lower formation of clonal colonies.

### Example 7

### Growth of ES Cells in Fully Defined Medium

ES cells are grown in a fully defined, albumin free, medium comprising DMEM F12 plus neurobasal medium (ratio 1:1) supplemented with insulin at 12.5 mg/L), apotransferrin 50 mg/L, progesterone 0.0099 mg/L, putrescine 8 mg/L and sodium selenite 2.5×10⁻⁶ mg/L.

Oct4GFP ES cells are passaged 6 times (cells passaged every 6-8 days) using cell dissociation buffer and replated after each passage at low density.

### Example 8

### Use of Serum-Free Medium and Transient Growth Factor Stimulation

ES cells are grown initially in HEScGRO or EScGRO medium (Examples 1 and 2). ES cells are plated at very low density, about 1000-10,000 cells on a 3.5 cm diameter plate and grown in the same medium.

It is envisaged that the numbers of undifferentiated ES cells will be enhanced, indicating increased proliferation or increased ES cell survival, or both.

The invention thus provides media and methods for self-renewal of ES cells of many species.

We claim:

1. A pluripotent cell culture medium comprising:
(a) insulin;
(b) progesterone; and
(c) transferrin and/or apotransferrin,
wherein the medium is free of serum and has an osmolarity in the range 260-270 Osm/kg.

2. A pluripotent cell culture medium comprising:
(a) 5 to 30 mg/L insulin; and
(b) 0.0005 to 0.05 mg/L progesterone,
wherein the medium is free of serum and has an osmolarity in the range 260-270 Osm/kg.

3. The pluripotent cell culture medium of claim 1 comprising:
(a) 5 to 30 mg/L insulin;
(b) 0.005 to 0.05 mg/L progesterone; and
(c) 25 to 75 mg/L or about 50 mg/L transferrin and/or apotransferrin.

4. The culture medium of claim 1, having an osmolarity of 263-266 Osm/kg.

5. The culture medium of claim 1, wherein the insulin, progesterone and transferrin and/or apotransferrin are human.

6. The culture medium of claim 1, wherein the transferrin and/or apotransferrin are recombinant.

7. The culture medium of claim 1, wherein the transferrin and/or apotransferrin are human.

8. The culture medium of claim 1, wherein the culture medium further comprises putrescine and/or sodium putrescine.2HCl.

9. The culture medium of claim 8, comprising from 3 to 20 mg/L putrescine and/or from 0.0005 to 0.1 mg/L sodium putrescine.2HCl.

10. The culture medium of claim 1, wherein the culture medium further comprises sodium selenite.

11. The culture medium of claim 11, comprising from 1×10⁻⁶ to 3×10⁻⁶, mg/L sodium selenite.

12. The culture medium of claim 1, wherein the culture medium is fully defined.

13. The culture medium of claim 1, wherein the culture medium has an osmolarity of about 265 Osm/kg.

14. The culture medium of claim 1, wherein the culture medium further comprises basic fibroblast growth factor (bFGF).

15. The culture medium of claim 14, wherein the bFGF is recombinant.

16. The culture medium of claim 14, wherein the bFGF is human.

17. The culture medium of claim 1, wherein the culture medium further comprises human serum albumin (HSA).

18. The culture medium of claim 17, wherein the HSA is recombinant.

19. The culture medium of claim 1, wherein the culture medium further comprises one or more of the following salt components:
(a) 100 to 200 mg/L calcium chloride anhydrous;
(b) 0.0005 to 0.0008 mg/L cupric sulphate;
(c) 0.05 to 1.00 mg/L ferric nitrate;
(d) 0.01 to 0.03 mg/L ferric sulphate;
(e) 200 to 400 mg/L potassium chloride;
(f) 10 to 20 mg/L magnesium chloride;
(g) 40 to 100 mg/L magnesium sulphate;
(h) 3000 to 7000 mg/L sodium chloride;
(i) 500 to 1500 mg/L sodium bicarbonate;
(j) 50 to 150 mg/L sodium phosphate monobasic;
(k) 20 to 50 mg/L sodium phosphate dibasic; and
(l) 0.1 to 0.3 mg/L zinc sulphate.

20. The culture medium of claim 1, wherein the culture medium further comprises one or more of the following:
(a) 3000 to 6000 mg/L D-glucose;
(b) 5 to 12 stet/L phenol red;
(c) 2000 to 4000 mg/L HEPES;
(d) 0.7 to 2.0 mg/L sodium hypoxanthine;
(e) 0.01 to 0.3 mg/L linoleic acid;
(f) 0.025 to 0.075 mg/L DL-68-thiotic acid; and
(g) 20 to 70 mg/L sodium pyruvate.

21. The culture medium of claim 1, wherein the culture medium further comprises one or more of the following amino acid components:
(a) 2 to 5 mg/L alanine;
(b) 50 to 200 mg/L arginine;
(c) 2 to 7 mg/L asparagine;
(d) 2 to 7 mg/L aspartic acid;
(e) 5 to 15 mg/L cysteine.H₂O;
(f) 10 to 20 mg/L cystine.2HCl;
(g) 200 to 400 mg/L glutamine;
(h) 2 to 7 mg/L glutamic acid;
(i) 20 to 30 mg/L glycine;
(j) 20 to 60 mg/L histidine;
(k) 50 to 100 mg/L isoleucine;
(l) 50 to 100 mg/L leucine;
(m) 100 to 150 mg/L lysine;
(n) 10 to 40 mg/L methionine;
(o) 25 to 75 mg/L phenylalanine;
(p) 5 to 20 mg/L proline;
(q) 15 to 50 mg/L serine;
(r) 50 to 100 mg/L threonine;
(s) 5 to 20 mg/L tryptophan;
(t) 40 to 90 mg/L tyrosine; and
(u) 50 to 100 73 mg/L valine.

22. The culture medium of claim 1, wherein the culture medium further comprises one or more of the following vitamin components:
(a) 0.001 to 0.003 mg/L biotin;
(b) 1 to 4 mg/L D-calcium panthenate;
(c) 4 to 8 mg/L choline chloride;
(d) 1 to 5 mg/L folic acid;
(e) 5 to 15 mg/L i-inositol;
(f) 1 to 5 mg/L niacinamide;
(g) 1 to 5 mg/L pyridoxine HCl;
(h) 0.1 to 0.5 mg/L riboflavine;
(i) 2 to 5 thiamine HCl;
(j) 0.1 to 0.4 mg/L thymidine; and
(k) 0.2 to 0.75 mg/L vitamin B12.

23. The culture medium of claim 1, wherein the culture medium is serum extract free and/or animal component free.

24. The culture medium of claim 1, wherein the culture medium is free of feeder cells and/or feeder cell extract.

25. The medium of claim 1, wherein the medium further comprises an agent that suppresses differentiation of pluripotent stem cells.

26. A method of culturing pluripotent cells so as to promote pluripotent cell self renewal, comprising maintaining the cells in the medium of claim 1.

27. The method of claim 26, wherein the pluripotent cells are human.

28. A method of culturing pluripotent cells, comprising:
(a) maintaining the cells in a pluripotent state in culture, optionally on feeders, in the presence of serum or an extract of serum;
(b) passaging the pluripotent cells at least once;
(c) withdrawing the serum or the serum extract from the medium and withdrawing the feeders if present, so that the medium is free of feeders, serum and serum extract; and
(d) subsequently maintaining the cells in a pluripotent state in the presence of the medium of claim 1.

29. The method of claim 28, wherein the culture comprises an agent that suppresses differentiation, preferably wherein the agent that suppresses differentiation is added to the culture medium at around the time that serum or serum extract is withdrawn.

30. The method of claim 28, wherein the pluripotent cells are human.

31. A method of obtaining a transfected population of pluripotent cells, comprising:
(a) transfecting pluripotent cells with a construct encoding a selectable marker;
(b) plating the pluripotent cells;
(c) culturing the pluripotent cells in the presence of the medium of claim 1; and
(d) selecting for the pluripotent cells that express the selectable marker.

32. The method of claim 31, wherein the pluripotent cells are human.

33. The method of claim 31, wherein the selectable marker encodes an antibiotic resistance or cell surface marker.

34. A method of culture of pluripotent cells, comprising:
(a) transferring an individual pluripotent cell to a culture vessel; and
(b) culturing the pluripotent cell in the presence of the medium of claim 1,
so as to obtain a clonal population of pluripotent cells, all of which are the progeny of a single pluripotent cell.

35. The method of claim 34, wherein the pluripotent cell is human.

36. The method of claim 34, wherein the culture vessel is an individual well on a plate.

37. A method of isolating a pluripotent cell comprising culturing tissue from an embryo, foetus or adult in the medium of claim 1.

38. A serum-free medium for self-renewal of pluripotent cells, comprising:
(a) a basal medium;
(b) insulin; and
(c) progesterone.

39. The serum-free medium of claim 38, further comprising one or more of the following components:
(a) transferrin and/or apotransferrin;
(b) putrescine and/or sodium putrescine;
(c) sodium selenite; and
(d) human bFGF.
